(19) United States
(12) Patent Application Publication
(10) Pub. No.: US 2003/0149409 A1
(43) Pub. Date: Aug. 7, 2003

(54) Urine collection & discharge device

(76) Inventor: Hsiang-Ting Lin, Taipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/060,190

(22) Filed: Feb. 1, 2002

(51) Int. Cl. A61F 5/44; A61F 13/15

(52) U.S. Cl. 604/349; 604/385.19; 604/385.09

(57) ABSTRACT

A urine collection and discharge device, specially designed for male patient with leakage problem, comprises a waistband, two suspenders and one pouch; two connection loops being provided on the waistband; one adjustment loop to each suspender; a buckling loop to one end of each suspender to fasten to the connection loop, and a clip to another end; the see-through pouch including an inner layer and an outer layer with its opening provided with pleated lip to be secured by the clip of the suspender for the patient to put his penis into the pouch.

### BACKGROUND OF THE INVENTION

(a) Field of the Invention

The present invention relates to a urine collection and discharge device for male leaky patient, and more particularly, to one that is well ventilated, infection free and allows a user for a limited outdoor activities.

(b) Description of the Prior Art

Urine collection devices generally available in the market for male leaky patients to wear are provided in the following types:

1) Sanitary Napkin for Male: it is composed of an inner layer which is permeable, an outer layer which is not permeable and an absorbent pad sandwiched by the inner and the outer layers; designed in a form similar to a sleeve, or to be folded into a shape of sleeve for the patient to put it on his penis before putting on his bikini briefs or boxer shorts. As the crotch is tightly attached to the sanitary napkin to prevent ventilation, the patient is vulnerable to get infection.

2) Adult diaper: an even wider coverage is required in use, thus leading to more concerns of infection.

3) Catheter pouch: as illustrated in FIG. 1 of the accompanying drawings, the pouch comprises an elastic band to restrict a sleeve (1) similar to a condom connected to its lower end a plastic catheter (2); the other end of the catheter (2) connected to a pouch (3) that can be fastened to the patient's lag; and the lower end of the pouch (3) is provided with a hose (4) to discharge the urine collected in the pouch (3). However, the pouch is found with defectives that adjustment for a proper elasticity of the sleeve (1) is difficult; either it feels discomfort if it gets too tight, or is easily to slip off if it gets too loosening, and that the penis for being confined in the sleeve (1) is also vulnerable to get infection.

### SUMMARY OF THE INVENTION

The primary purpose of the present invention is to provide a urine collection and discharge device comprising a waistband, two suspenders, and a pouch. Each suspender is provided with an adjustment loop, one end is provided with a bucking loop to hook up a connection loop of the waistband and the other end is provided with a clip. The pouch is composed of two transparent layers with an opening having a pleated lip for both of the suspender to hook up. The penis of the patient can be comfortably placed into the pouch through the opening to collect urine so that the patient feels free to engage in some limited outdoor activities. Furthermore, the present invention giving good ventilation to the penis of the patient is practically infection free.

### BRIEF DESCRIPTION OF THE DRAWINGS

FIG. 1 is a perspective view of a prior art of a catheter;

FIG. 2 is a perspective view of a first preferred embodiment of the present invention;

FIG. 3 is an exploded view of a combination of an inner layer and outer layer of a pouch of the first preferred embodiment of the present invention;

FIG. 3A is a perspective view of the combination of the pouch of the first preferred embodiment of the present invention;

FIG. 3B is a perspective view of a lip of the pouch of the first preferred embodiment of the present invention;

FIG. 4 is a schematic view showing the first preferred embodiment of the present invention is in use;

FIG. 5 is a perspective view of a second preferred embodiment of the present invention;

FIG. 6 is a schematic view of the second preferred embodiment of the present is in use;

FIG. 7 is a perspective view of a third preferred embodiment of the present invention; and

FIG. 8 is a schematic view of the third preferred embodiment of the present is in use;

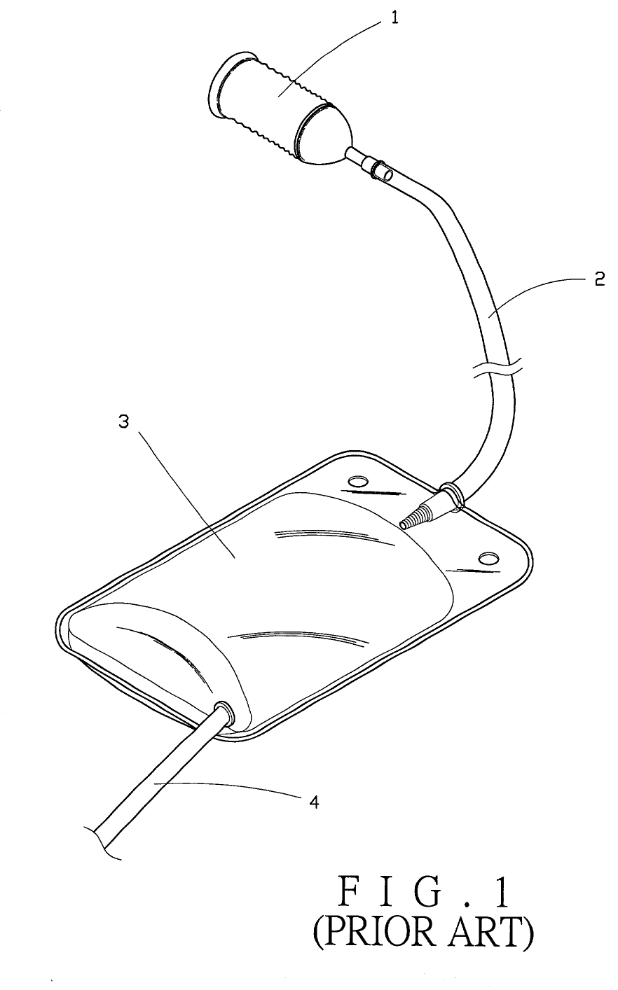

### DETAILED DESCRIPTION OF THE PREFERRED EMBODIMENTS

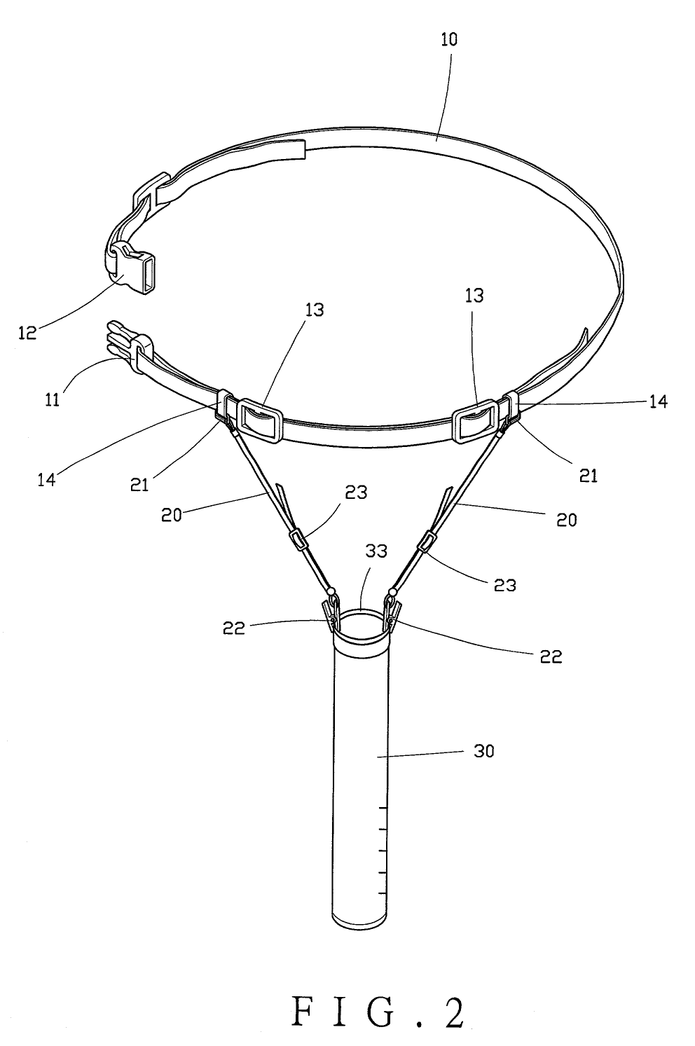

As illustrated in FIG. 2, the present invention comprises a waistband (10), two spenders (20) and a pouch (30).

Two ends of the waistband (10) are respectively provided with a male buckle (11) and a female buckle (12). Two fixation loops (13) are provided at where appropriately, and it is preferred that both loops (13) shall be at least 15 cm away from each other. On relatively farther side of each said fixation loop (13) is provided with a connection loop (14). The fixation loop (13) is a square ring having at its center provided with a tongue [not illustrated] for the purpose of adjusting the length of the waistband (10) depending on the patient's waist size.

One end of each said suspender (20) is provided with a buckling loop (21) to fasten to the connection loop (14) while the other end is incorporated with a clip (22). Each said suspender (20) is provided with an adjustment loop (23) in the configuration of a square ring having at its center provided with stick [not illustrated] for the suspender (20) to pierce into it for adjusting the length of suspender (20).

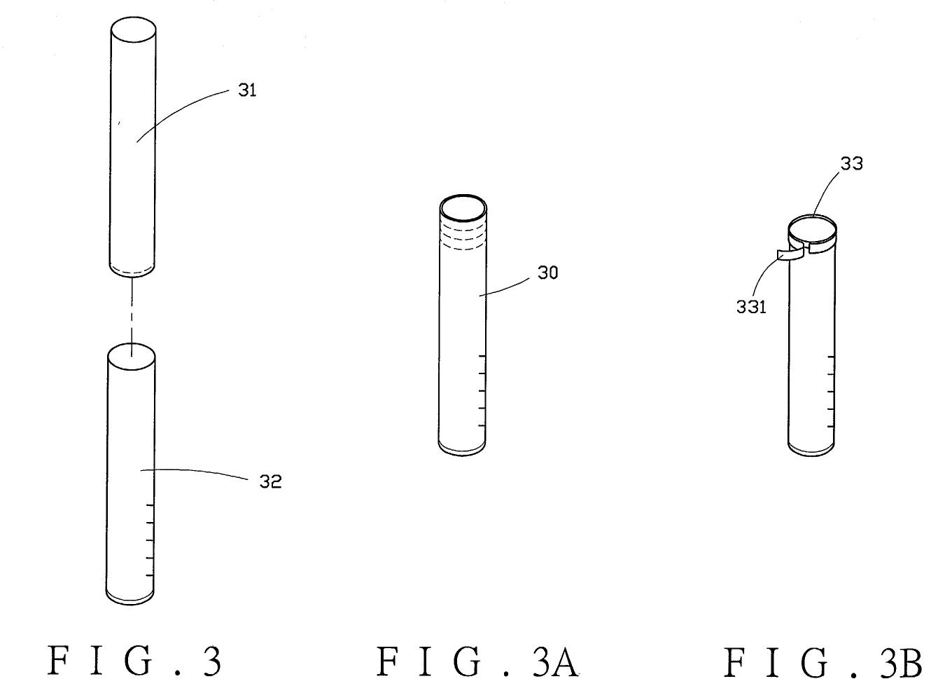

As illustrated in FIG. 3, the pouch (30) is a tubular pouch comprising a see-through inner layer (31) placed into a tubular see-through outer layer (32) [both of the inner layer (31) and the outer layer (32) is each in diameter of approximately 6.5 cm and in length of approximately 37.5 cm; a soft cotton fabric is adhered to the outer layer (32) to achieve natural and comfortable touch of the pouch (30) to the skin]. The opening of the inner layer (31) is flushed with that of the outer layer (32). Now referring to FIGS. 3A and 3B, the openings of each of the inner layer (31) and the outer layer (32) are pleated inside out for three times for a length of approximately 0.7 cm each pleat to form a lip (33). A transparent adhesive tape (331) is then used to hold the lip (33) in position to facilitate the attachment of the clip (22).

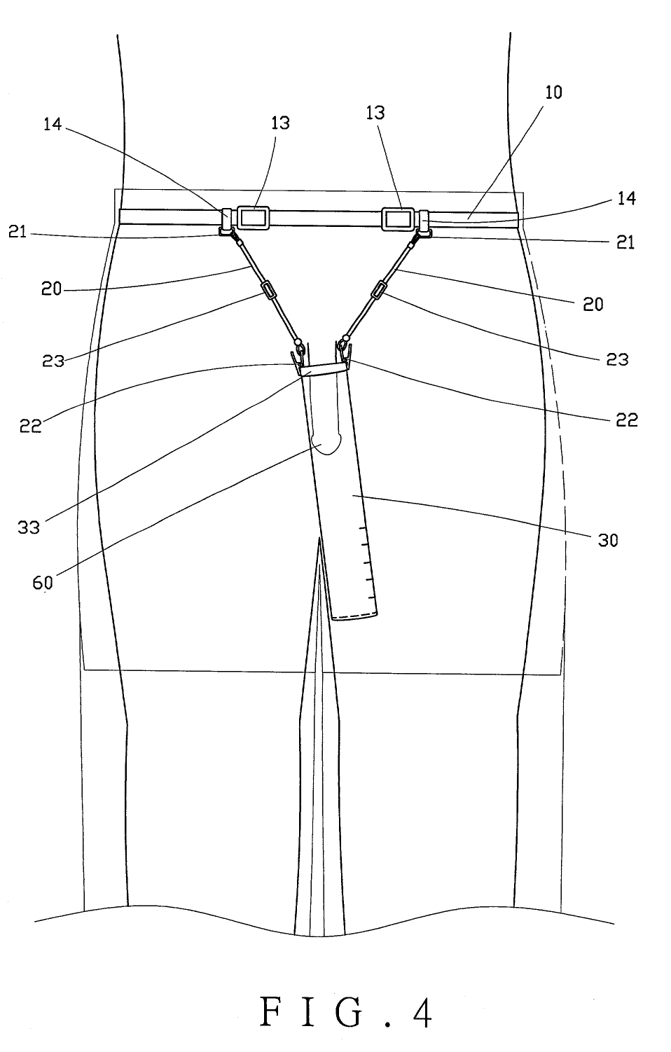

As illustrated in FIG. 4 when the first preferred embodiment is in use, the waistband (10) is surrounded the waist of the patient at the lowest waist line by means of the male and the female buckles (11, 12). The fixation loops (13) are used to adjust the length of the waistband (10) so that the pouch (30) is located in the center of the front of the patient for the patient to put his penis (60) into the pouch (30). Each clip (22) of the suspender (20) is tightly secured to both sides of the lip (33) at the opening of the pouch (30). The adjustment loop (23) is used to adjust the length of each suspender (20) to where is tight enough without compromising the comfort of the patient. Finally, the patient puts on his underwear and trousers and makes sure that the pouch (30) naturally suspends in the trousers.

Once the urine collected in the pouch (30) reaches a certain level, preferred not to exceed the top scale (one third of the pouch), the patient could dispose it in the toilet and replace with a new one. Alternatively, in case of the absence of replacement or emergency, simply pour out the urine then wear the same pouch (3). As the urine accumulates in the pouch (30), the pouch (3) gets heavier and the connection loop (14) connecting the suspender (2) slides toward the center in front of the patient. In such case, the fixation loop (13) will retain the connection loop (14) from moving towards the center in front of the patient.

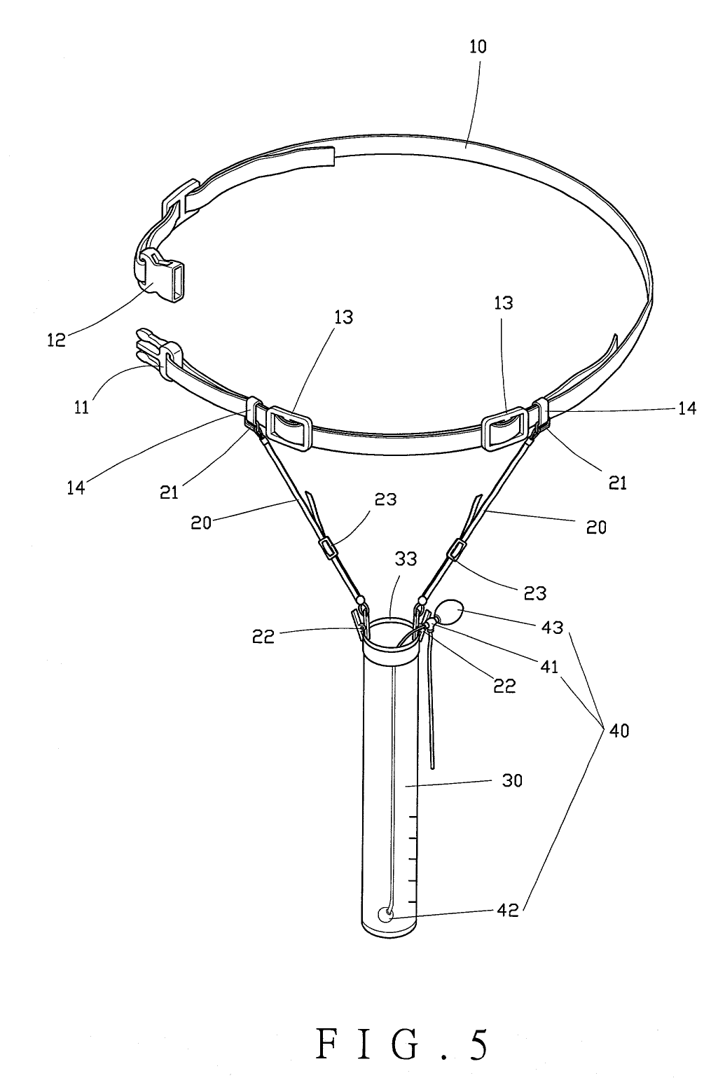

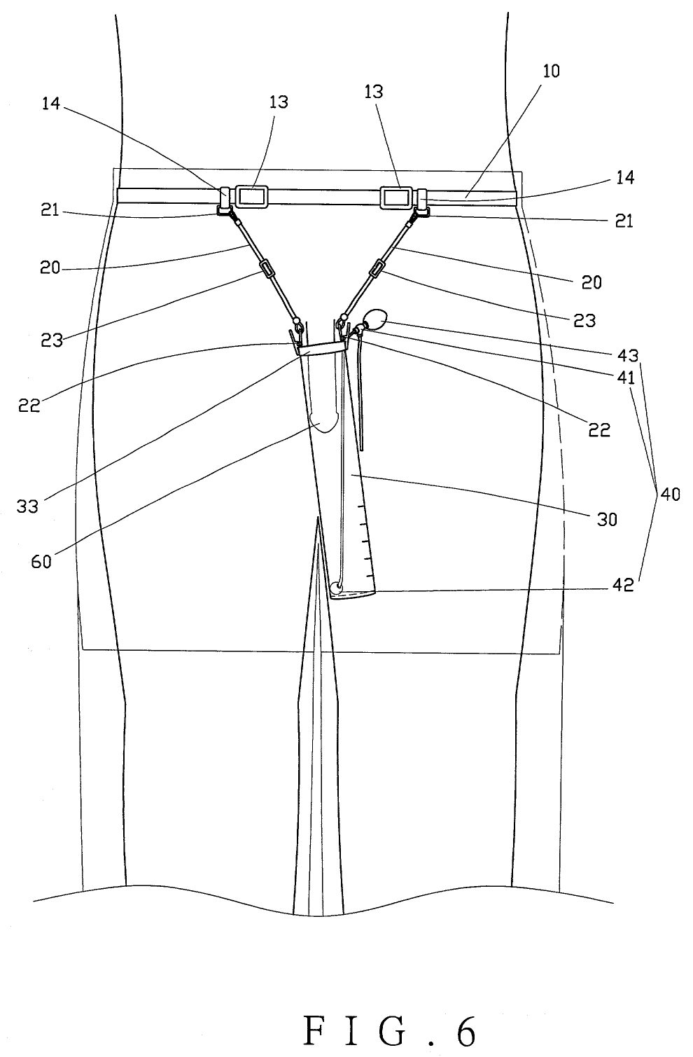

Now referring to FIGS. 5 and 6, a second preferred embodiment of the present invention is adapted with a pumping device (40). The pumping device (40) is composed of a pumping hose (41) having a ball (42) at its bottom and a manually operating pump (43) at its top. When the second preferred embodiment is in use, the waistband (10) is surrounded the waist of the patient at the lowest waist line by means of the male and the female buckles (11, 12). The pumping device (4) is placed into the pouch (30) leaving the manually operating pump (43) to be out of the opening of the pouch (30) so that the ball (42) of the pumping device (4) contacts the bottom of the pouch (30) by the weight of the ball (42). Then, the patient puts his penis (60) into the pouch (30). Each clip (22) of the suspender (20) is tightly secured to both sides of the lip (33) at the opening of the pouch (30). The adjustment loop (23) is used to adjust the length of each suspender (20) to where is tight enough without compromising the comfort of the patient. Finally, the patient puts on his underwear and trousers and makes sure that the pouch (30) naturally suspends in the trousers.

Once the urine collected in the pouch (30) reaches a certain level, preferred not to exceed one third of the porch, the patient (3) just goes to the toilet and press the manually operating pump (43) of the pumping device (40) to pump out the urine accumulated in the pouch (30). It is preferred that the pumping device (40) be made a disposal one.

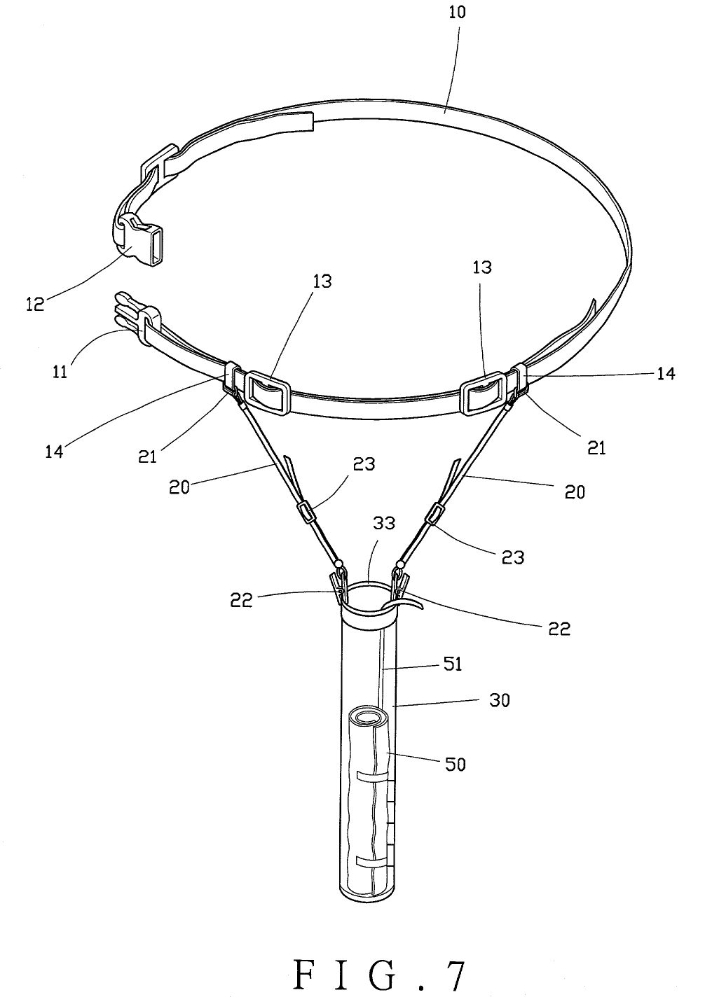

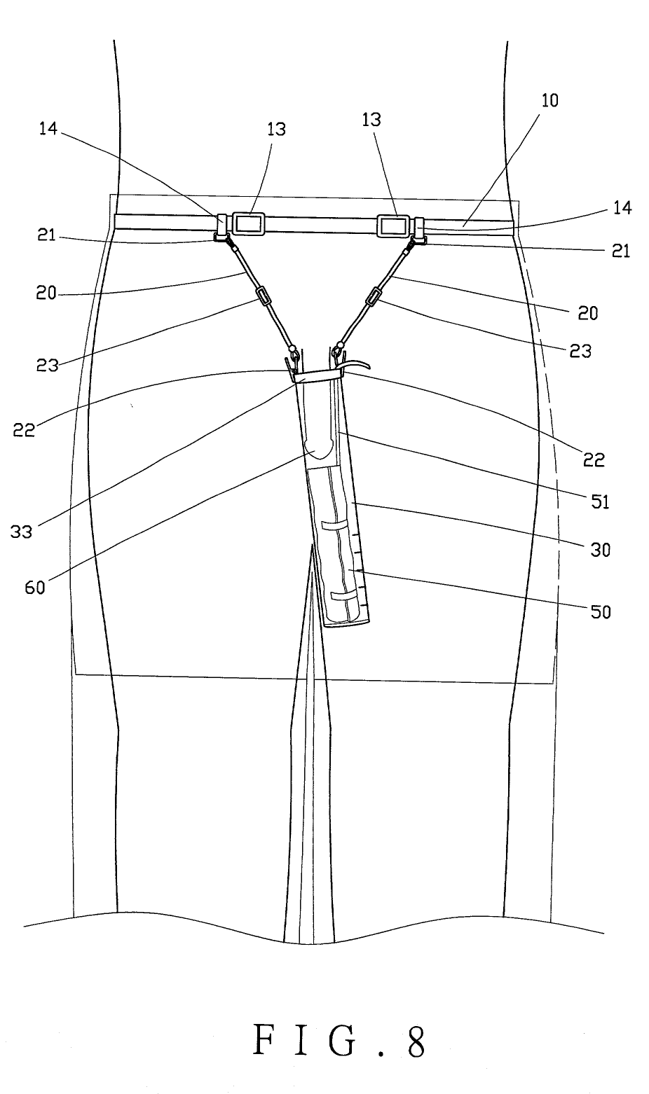

Now referring to FIGS. 7 and 8, a light, highly absorbent sanitary napkin (50) is adapted to a third preferred embodiment of the present invention. Wherein, the sanitary napkin (50) is wound into a roll provided with a string (51) to pull. When in use, the patient wears the waistband (10) around his waist by means of the male and the female buckles (11, 12) to leave the pouch (30) at the center in front of him. Then the sanitary napkin (50) is placed into the pouch (30) leaving the string (51) to rest for a proper section outside the opening of the pouch (30) before putting the penis (60) into the pouch (30). Finally, each clip (22) of the suspender (20) is secured tightly to both sides of the lip (33) of the pouch (30) while using the adjustment loop (23) to adjust the length of the suspender (20) to such extent allowing slight tightness without compromising the comfort of the patient. The patient finally puts his underwear and trousers and makes sure that the pouch (30) naturally suspends in the trousers.

The urine collected by the pouch (30) is absorbed by the sanitary napkin (50) so to eliminate odor and avoid urine from being disturbed. The sanitary napkin (50) is disposed of together with the residual urine or simply the soaked up sanitary napkin is disposed of pulling out the sanitary napkin (50) by the string (51), then place a new sanitary napkin into the pouch (30). However, it is recommended that the pouch (30) shall be replaced with a new one every day.

I claim:

1. A urine collection and discharge device comprising a waistband, two suspenders and a pouch; both ends of said waistband being respectively connected with a male buckle and a female buckle to be engaged for a patient to wear around his waist characterized by that:
two connection loops being provided on said waistband, one adjustment loop is each provided to said suspenders; one end of each suspender being provided with a buckling loop to fasten to said connection loop; and the other end of each suspender being provided with a clip to clamp both sides of an opening of said pouch.

2. A urine collection and discharge device as claimed in claim 1, wherein, two fixation loops are provided on the waistband, and said connection loop is each respectively provided on the relatively farther sides of said two fixation loops.

3. A urine collection and discharge device as claimed in claim 1, wherein, the opening of said pouch is provided with a lip by repeated pleated an upper edge of said pouch.

4. A urine collection and discharge device as claimed in claim 1, wherein, the pouch comprises a see-through inner layer in tubular shape and a see-through outer layer in tubular shape adhered a soft cotton fabric on its outer surface.

5. A urine collection and discharge device as claimed in claim 1, wherein, a pumping device is provided in the pouch, said pumping device comprising a pumping hose having a ball at its bottom and a manually operating pump at its top; both of said pumping hose and said ball being placed in said pouch and said manually operating pump being left outside the opening of said pouch.

6. A urine collection and discharge device as claimed in claim 1, wherein, a light, highly absorbent sanitary napkin is placed in said pouch, said sanitary napkin being wound into a roll and a string attached to its top.
